(19) United States
(12) Patent Application Publication
(10) Pub. No.: US 2026/0009774 A1
(43) Pub. Date: Jan. 8, 2026

(54) SENSOR INCLUDING AN ANODIZED POROUS LAYER AND METHOD OF FORMING A SENSOR

(71) Applicant: Microchip Technology Incorporated, Chandler, AZ (US)

(72) Inventors: Patrick McFarland, Gilbert, AZ (US); Steve Nagel, Chandler, AZ (US); Bomy Chen, Newark, CA (US); Arthur B. Eck, Gilbert, AZ (US)

(73) Assignee: Microchip Technology Incorporated, Chandler, AZ (US)

(21) Appl. No.: 18/890,469

(22) Filed: Sep. 19, 2024

Related U.S. Application Data
(60) Provisional application No. 63/667,758, filed on Jul. 4, 2024.

(51) Int. Cl. G01N 33/00; C25D 11/18

(52) U.S. Cl. CPC G01N 33/0027; C25D 11/18

(57) ABSTRACT

A method includes forming a metal cathode on a substrate, anodizing an outer surface of the metal cathode to form an anodized porous layer, and forming a metal anode over the anodized porous layer, wherein the anodized porous layer defines a dielectric layer between the metal anode and the metal cathode, and wherein the metal anode, the anodized porous layer, and the metal cathode define a sensor.

### RELATED APPLICATION

This application claims priority to commonly owned U.S. Provisional Patent Application No. 63/667,758 filed Jul. 4, 2024, the entire contents of which are hereby incorporated by reference for all purposes.

### TECHNICAL FIELD

The present disclosure relates to environmental sensors, and more particularly, to a sensor including an anodized porous layer, and method of forming such sensor.

### BACKGROUND

There is a large market demand for integrated environmental sensors (i.e., environmental sensors at an integrated circuit scale), for example in industries including smoke/carbon monoxide (CO) monitoring, agriculture, mining and construction, without limitation. Many industries and applications need sensors in high volume and with high reliability. Integrated sensors that measure the environment are typically exposed to the environment, and are therefore subject to environmental contamination, e.g., ionic contamination. In addition, conventional integrated environmental sensors are often large, expensive, and unreliable.

There is a need for improved sensors and methods of forming sensors.

### SUMMARY

The present disclosure provides sensors, e.g., integrated circuit (IC) sensors, including an anodized porous layer between a cathode and an anode, and methods for forming such sensors. Sensors as disclosed herein may be relatively reliable, inexpensive, and resistant to contamination (e.g., as compared with conventional sensors), and disclosed methods of forming such IC sensors may be relatively low cost and high yield (e.g., as compared with conventional sensors methods).

Some examples provide an IC sensor including a cathode, an anode, and a porous oxide layer defining a dielectric/insulator between the cathode and anode, wherein the IC sensor changes resistance or capacitance based on the concentration of one or more environmental conditions, e.g., humidity, gas, smoke, etc. Such sensors may be tunable during manufacturing, for example for detecting different parameters (e.g., smoke, CO, humidity, a particular gas or gasses, etc.) based on relevant environmental conditions. In some examples, the cathode may be formed with a 3D (non-planar) outer surface to thereby define a 3D nanoporous dielectric layer, wherein the anode may also have a 3D structure. In other examples, the cathode may be formed as a planar structure to thereby define a planar nanoporous dielectric layer formed thereon.

Some examples provide a sensor including an aluminum cathode formed on a substrate, a nanoporous aluminum oxide layer formed by anodizing an outer surface of the aluminum cathode, and an aluminum anode formed over the aluminum cathode. In some example, the nanoporous aluminum oxide layer may be at least partially filled with at least one substance, e.g., tin oxide, nickel oxide, titanium oxide, or other oxide. In some examples, the aluminum cathode may be formed as a 3D structure with a 3D outer surface to thereby define a 3D nanoporous aluminum oxide layer formed thereon, wherein the overlying aluminum anode may also have a 3D structure. The 3D aluminum cathode may increase the area of the resulting 3D nanoporous aluminum oxide layer (e.g., as compared with structure having a planar cathode), which may increase the sensitivity of the resulting sensor. In other examples, the aluminum cathode may be formed with a planar or substantially planar outer surface to thereby define a planar or substantially planar nanoporous aluminum oxide layer.

One aspect provides a method including forming a metal cathode on a substrate, anodizing an outer surface of the metal cathode to form an anodized porous layer, and forming a metal anode over the anodized porous layer. The anodized porous layer defines a dielectric layer between the metal anode and the metal cathode. The metal anode, the anodized porous layer, and the metal cathode define a sensor.

In some examples, the method includes at least partially filling the anodized porous layer with at least one substance, for example at least one oxide, before forming the metal anode over the anodized porous layer.

In some examples, the metal cathode comprises a three-dimensional metal cathode having a three-dimensional outer surface including different areas extending in different planes, and the anodized porous layer comprises a three-dimensional porous layer.

In some examples, forming the three-dimensional metal cathode comprises forming a metal and selectively etching portions of the metal layer within an outer lateral perimeter of the metal layer to define a three-dimensional structure.

In some examples, forming the three-dimensional metal cathode comprises forming a metal and selectively etching portions of the metal layer to define an array of spaced apart cathode elements.

In some examples, a thickness of the three-dimensional metal cathode varies by at least 25% or at least 0.5 μm at different locations across a lateral footprint of the cathode.

In some examples, forming the metal cathode comprises forming an aluminum cathode, and anodizing the outer surface of the metal cathode to form the anodized porous layer comprise anodizing an outer surface of the aluminum cathode to form a nanoporous aluminum oxide layer.

In some examples, the method includes at least partially filling the nanoporous aluminum oxide layer with at least one substance, e.g., at least one oxide, before forming the metal anode.

In some examples, the method includes at least partially filling the nanoporous aluminum oxide layer with at least one of tin oxide, nickel oxide, or titanium oxide before forming the metal anode.

In some examples, forming the metal cathode comprises forming a three-dimensional aluminum cathode having a three-dimensional outer surface including different areas extending in different planes, and anodizing the outer surface of the metal cathode to form the anodized porous layer comprise anodizing the three-dimensional outer surface of the aluminum cathode to form a three-dimensional nanoporous aluminum oxide layer.

In some examples, forming the three-dimensional aluminum cathode includes depositing an aluminum layer and selectively etching portions of the aluminum layer within an outer lateral perimeter of the aluminum layer to define a three-dimensional structure.

In some examples, forming the metal anode over the anodized porous layer comprises forming a three-dimensional aluminum anode over the three-dimensional nanoporous aluminum oxide layer by depositing an aluminum layer over the three-dimensional nanoporous aluminum oxide layer, and selectively etching portions of the deposited aluminum layer, wherein the three-dimensional nanoporous aluminum oxide layer acts as an etch stop.

One aspect provides a sensor including a metal cathode formed on a substrate, an anodized porous layer formed on an outer surface of the metal cathode, and a metal anode formed over the anodized porous layer.

In some examples, the sensor includes at least one substance, e.g., at least one oxide, at least partially filling the anodized porous layer.

In some examples, the metal cathode comprises a three-dimensional metal cathode having a three-dimensional outer surface including different areas extending in different planes, and the anodized porous layer comprises a three-dimensional porous layer.

In some examples, the metal cathode comprises an aluminum cathode, and the anodized porous layer comprises a nanoporous aluminum oxide layer.

In some examples, the metal cathode comprises a three-dimensional aluminum cathode having a three-dimensional outer surface including different areas extending in different planes, and the anodized porous layer comprises a three-dimensional nanoporous aluminum oxide layer. In some examples, the sensor includes at least one oxide at least partially filling the three-dimensional nanoporous aluminum oxide layer.

One aspect provides a sensor system, including (a) a sensor including an aluminum cathode formed on a substrate, a nanoporous aluminum oxide layer formed on an outer surface of the aluminum cathode, and an aluminum anode formed over the nanoporous aluminum oxide layer, and (b) sensor circuitry connected to the cathode and the anode to measure at least one of a resistance or a capacitance of the sensor.

In some examples of the sensor system, the aluminum cathode comprises a three-dimensional structure having a three-dimensional outer surface including different areas extending in different planes, and the nanoporous aluminum oxide layer has a three-dimensional structure.

In some examples, the sensor system includes at least one oxide at least partially filling the nanoporous aluminum oxide layer.

### BRIEF DESCRIPTION OF THE DRAWINGS

Example aspects of the present disclosure are described below in conjunction with the figures, in which:

FIG. 1 shows a flowchart of an example method of forming a sensor including an anodized porous layer between a cathode and an anode;

FIGS. 2-5B illustrate an example method of forming an example sensor including a 3D nanoporous aluminum oxide layer between a 3D aluminum cathode and a 3D aluminum anode;

FIG. 6 shows an example 3D aluminum cathode formed as a contiguous structure;

FIGS. 7-9 illustrate an example method of forming a sensor with a planar cathode and anodized porous layer; and

FIG. 10 shows an example sensor system including the example sensor shown in FIGS. 5A-5B and sensor circuitry connected to the sensor.

It should be understood that the reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

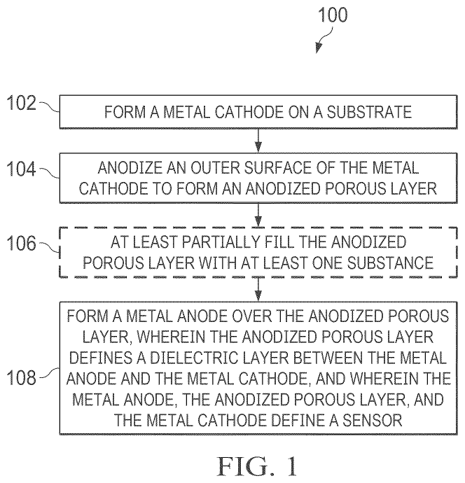

FIG. 1 shows a flowchart of an example method 100 of forming a sensor, in particular a IC sensor including an anodized porous layer between a cathode and an anode. At 102, a metal cathode is formed on a substrate. In some examples, the metal cathode comprises aluminum. In other examples, the metal cathode may comprise titanium or other metal. In some examples, the substrate (for example in the form of a wafer) may include SiO₂, silicon-rich SiO₂, borophosphosilicate glass (BPSG), or other dielectric material.

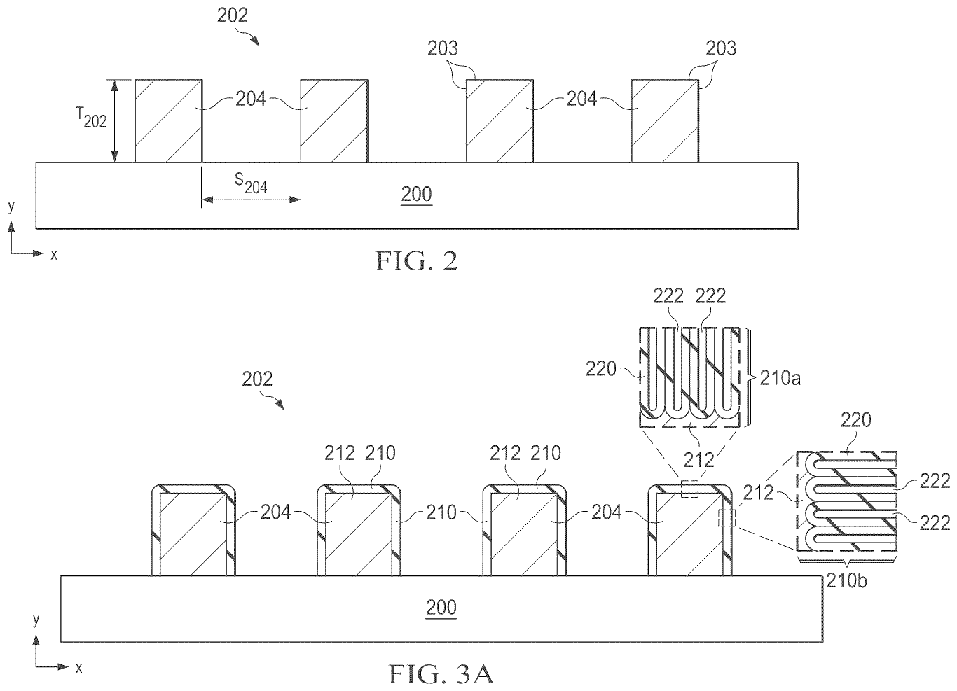

The metal cathode may be formed as a three-dimensional (3D) structure (i.e., a 3D cathode) or alternatively as a planar structure (i.e., a planar cathode). As used herein, a 3D structure refers to a structure having a thickness that varies by at least 25% or at least 0.5 μm at different locations across the lateral footprint of the structure. For example, a 3D cathode may include an array of vertically-projecting components, e.g., formed by depositing a metal layer and etching selected areas within an outer lateral perimeter of the metal layer to partially or fully remove the thickness of the metal layer in the selected areas. In some example, e.g., as shown in FIGS. 2-5B discussed below, a 3D cathode may include an array of spaced-apart cathode elements. A 3D cathode may have a 3D outer surface including different surface areas extending in different planes (e.g., including vertically-extending planes, horizontally-extending planes, and/or other angled planes) at locations within the outer lateral perimeter of the 3D cathode (i.e., not only at the outer lateral edges of the 3D cathode).

In contrast, as used herein a planar structure refers to a structure having a thickness that varies by less than 25% and less than 0.5 μm across the lateral footprint of the structure. For example, a planar cathode may comprise a metal layer formed by depositing a metal layer using a sputtering or evaporation process.

The 3D outer surface of the cathode may provide an increased surface area to be anodized, e.g., as discussed at 104, which may provide an anodized porous layer (defining the dielectric of the sensor) having an increased area, as compared with an anodized porous layer formed on a planar cathode.

At 104, an anodization process is performed to anodize exposed outer surface(s) of the metal cathode to form an anodized porous layer over (on) the metal cathode, wherein the anodized porous layer may define the dielectric of the sensor being formed. In some examples, the anodization process may include clamping the substrate (e.g., wafer) to define a conductive contact, and running a current through a bath (e.g., a sulfuric acid bath), for example a similar process as used for anodizing aluminum bike parts, etc. In examples in which the cathode is formed from aluminum, the anodized porous layer may comprise a nanoporous aluminum oxide layer. The nanoporous aluminum oxide layer may include an array of parallel pores (nanopores) extending inwardly from the outer surface of the anodized porous layer, e.g., wherein the pores form a honeycomb-like structure.

At 106, an optional process is performed to at least partially fill the anodized porous layer (e.g., nanoporous aluminum oxide layer in the case of an aluminum cathode) with at least one substance, referred to herein as a filler. In some examples, the filler may comprise an oxide (e.g., tin oxide, nickel oxide, or titanium oxide), or other compound. The filler may be selected to enhance the operation of the sensor being formed. For example, the filler may comprise a substance the changes in resistance or capacitance based on the presence of certain environmental substance or condition(s), for example, humidity or gas (e.g., CO, CO₂, or methane), or other environmental substance or condition. In some examples, the filler may increase the sensor's specificity for detecting certain environmental substance(s) or condition(s). In some examples, different areas of the anodized porous layer may be at least partially filled with different fillers, for example for detection of different environmental substances or conditions using the same sensor. In other examples, the introduction of filler(s) may be omitted, i.e., the optional process at 106 may be omitted.

At 108, a metal anode is formed over the anodized porous layer. The anodized porous layer (e.g., nanoporous aluminum oxide layer) defines a dielectric layer between the metal anode and the metal cathode. The metal anode, the anodized porous layer (with optional filler substance(s)), and the metal cathode collectively define a sensor. In some examples, the metal anode may be formed as a 3D anode over a 3D anodized porous layer by (a) depositing a metal (e.g., aluminum) layer over the 3D anodized porous layer (e.g., nanoporous aluminum oxide layer) and (b) selectively etching portions of the deposited metal layer, wherein the 3D anodized porous layer acts as an etch stop.

FIGS. 2-5B illustrate an example method of forming an example sensor including a 3D nanoporous aluminum oxide layer between a 3D aluminum cathode and a 3D aluminum anode. It should be understood the example method shown in FIGS. 2-5B may be similarly performed using other materials, e.g. using other metal(s) than aluminum to form the cathode, anodized porous layer, and/or anode.

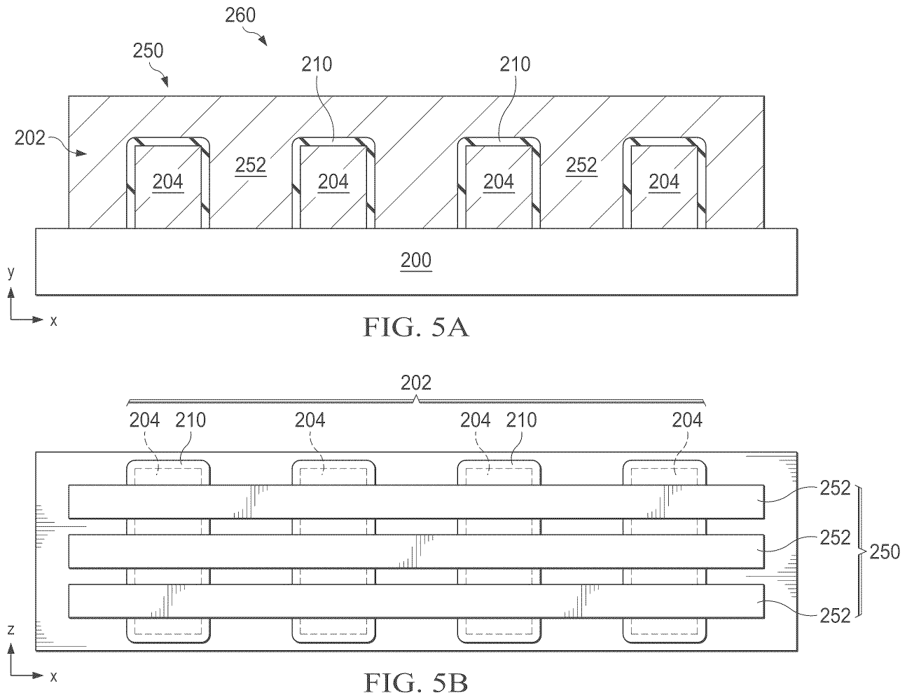

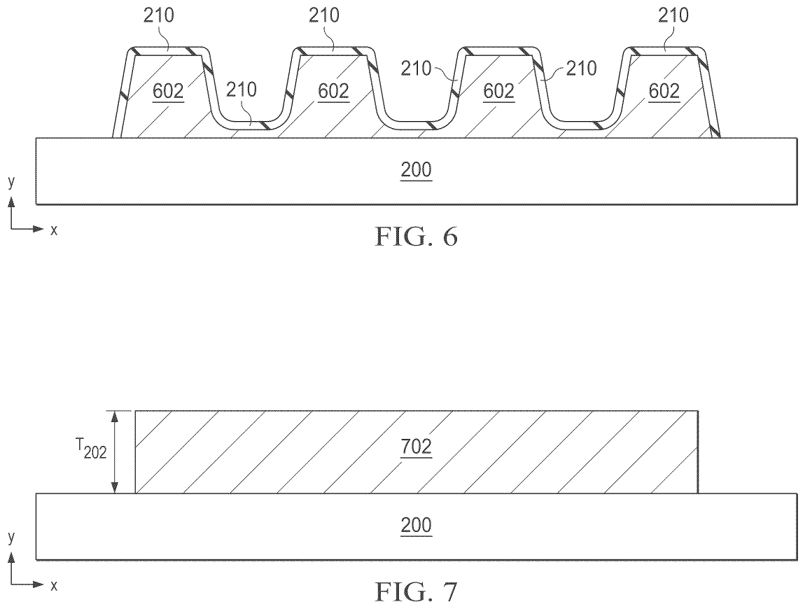

As shown in FIG. 2 (cross-sectional side view), a 3D aluminum cathode 202 is formed on a substrate 200. The substrate 200 may be embodied as a wafer or other form of substrate, and may include a suitable dielectric, for example SiO₂, silicon-rich SiO₂, BPSG, or any other suitable material. The 3D aluminum cathode 202 has a 3D outer surface 203 including different areas extending in different planes, including the x-y plane, y-z plane, and x-z plane (the z axis is explicitly shown in FIG. 5B). In this example, the aluminum cathode 202 includes an array of spaced-apart cathode elements 204, which may be electrically connected to each other (i.e., out of the plane of FIG. 1). In other examples, e.g., as shown in FIG. 6 discussed below, a 3D cathode may be formed as a contiguous structure, e.g., with undulations or other three-dimensional (non-planar) shape.

In some examples, the aluminum cathode 202 may be formed by depositing an aluminum layer (e.g., by a sputtering or evaporation process), and selectively etching away portions of the aluminum layer, leaving the cathode elements 204. The etch process may include a photoresist or hard mask, and the etch may be a wet etch or plasma etch, for example, wherein the substrate 200 may act as an etch stop.

In some examples, the aluminum cathode 202 may have a vertical thickness T₂₀₂ in the range of 0.8-2.0 μm (e.g., wherein a double aluminum deposition may be used for larger thickness), and a lateral spacing thickness S₂₀₂ between adjacent cathode elements 204 may be in the range of 0.25-1.0 μm. It should be understood these dimensions are examples only.

In other examples, titanium or other metal may be used instead of aluminum, although such examples may involve increased costs.

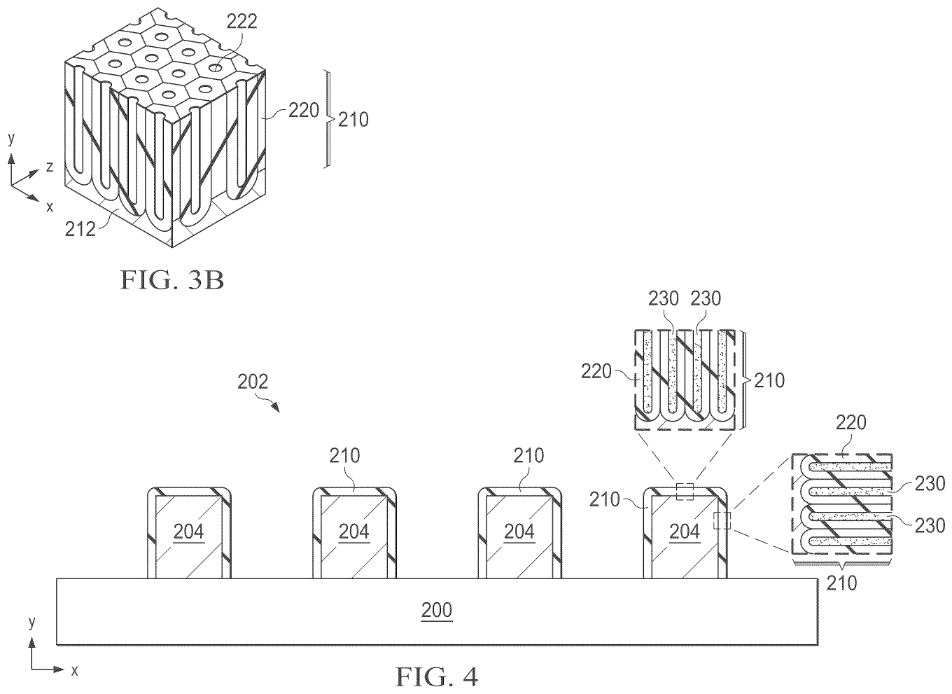

As shown in FIG. 3A (cross-sectional side view), the 3D outer surface 203 of the 3D aluminum cathode 202 may be anodized to form a 3D nanoporous aluminum oxide layer 210 over the underlying aluminum 212 of the of respective cathode elements 204, wherein the 3D nanoporous aluminum oxide layer 210 may extend over exposed surfaces of respective cathode elements 204, e.g., including top surfaces and sidewall surfaces of respective cathode elements 204. In some examples, the anodization process may include clamping the substrate 200 (e.g., wafer) to define a conductive contact, and running a current through a bath (e.g., a sulfuric acid bath), for example a similar process as used for anodizing aluminum bike parts, etc.

FIG. 3A shows a magnified view of two selected areas of the 3D nanoporous aluminum oxide layer 210, indicated at 210a (a horizontally-extending area of aluminum oxide layer 210) and 210b (a vertically-extending area of aluminum oxide layer 210). In addition, FIG. 3B shows a three-dimensional view of a selected area of aluminum oxide layer 210, e.g., corresponding with example area 210a. As shown, the nanoporous aluminum oxide layer 210 comprises an aluminum oxide region 220 including an array of parallel pores (nanopores) 222 extending inwardly from the outer surface, e.g., forming a honeycomb-like structure. The nanoporous aluminum oxide layer 210 may also be referred to as porous aluminum oxide (PAO) or a nanoporous aluminum membrane (NPAM). In some examples, respective nanopores 222 may have a diameter in the range of 100-1000 Å, and a depth-to-diameter aspect ratio in the range of 2:1 to 10:1.

As shown in FIG. 4 (cross-sectional side view), the nanoporous aluminum oxide layer 210 (in particular, nanopores 222) may be at least partially filled with at least one filler material 230, for example, another oxide (e.g., tin oxide, nickel oxide, or titanium oxide), or other compound. The filler material 230 may be selected to enhance the operation of the sensor being formed. For example, the filler material 230 may comprise a substance the changes in resistance or capacitance based on the presence of certain environmental substance or condition(s), for example, humidity or gas (e.g., CO, CO2, or methane), or other environmental substance or condition. In some examples, the filler material 230 may increase the specificity of detection of certain environmental substance or condition(s). In some examples, different areas of the nanoporous aluminum oxide layer 210 may be at least partially filled with different filler materials 230, for example for detection of different environmental substances or conditions using the same sensor. In other examples, the introduction of filler material(s) 230 shown in FIG. 4 may be omitted.

As shown in FIG. 5A (cross-sectional side view) and FIG. 5B (top view) collectively, a 3D aluminum anode 250 is formed over the 3D nanoporous aluminum oxide layer 210, to define a sensor 260 including the 3D aluminum anode 250, 3D aluminum cathode 202, and 3D nanoporous aluminum oxide layer 210. In this example, the aluminum anode 250 includes an array of spaced-apart anode elements 252, which may be electrically connected to each other (not shown). In other examples, the aluminum anode 250 may be formed as a contiguous structure.

In some examples, the 3D aluminum anode 250 may be formed in a similar manner as the 3D aluminum cathode 202. For example, the 3D aluminum anode 250 may be formed by depositing an aluminum layer (e.g., by a sputtering or evaporation process), and selectively etching away portions of the aluminum layer, leaving the array of anode elements 252. The etch process may include a photoresist or hard mask, and the etch may be a wet etch or plasma etch, for example, wherein the nanoporous aluminum oxide layer 210 may act as an etch stop.

FIG. 6 shows an example 3D aluminum cathode 602 formed as a contiguous structure having an undulating outer surface defining respective peaks and valleys, and a 3D nanoporous aluminum oxide layer 210 formed thereon (by anodizing the aluminum cathode 602). The 3D aluminum cathode 602 may be formed in the example process shown in FIGS. 2-5B, e.g., as an alternative to the example 3D aluminum cathode 202 discussed above. Like the 3D aluminum cathode 202 discussed above, the example aluminum cathode 602 may be formed by depositing an aluminum layer (e.g., by a sputtering or evaporation process), and selectively etching portions of the aluminum layer, e.g., without etching down to the substrate 200.

FIGS. 7-9 illustrate an example method of forming a sensor with a planar cathode and anodized porous layer. The example method shown in FIGS. 7-9 may be similar to the example method shown in FIGS. 2-5B and discussed above, except a planar aluminum cathode 702 may be formed instead of the 3D dimensional aluminum cathode 202 discussed above.

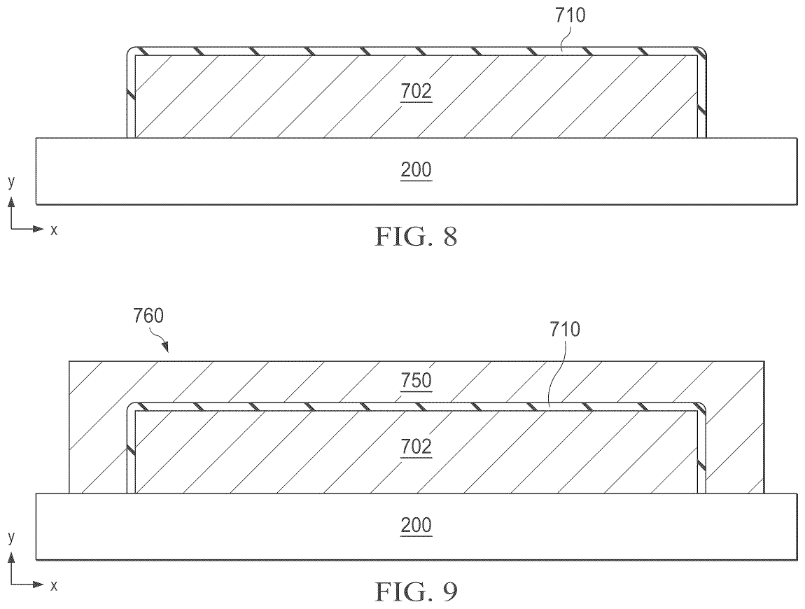

As shown in FIG. 7, a planar aluminum cathode 702 is formed on a substrate 200. As shown in FIG. 8, an outer surface of the aluminum cathode 702 may be anodized to form a nanoporous aluminum oxide layer 710 on the aluminum cathode 702. In some examples, the nanoporous aluminum oxide layer 210 (in particular, nanopores in the nanoporous aluminum oxide layer) may be at least partially filled with at least one filler material 230, for example, another oxide (e.g., tin oxide, nickel oxide, or titanium oxide), or other compound, e.g., as discussed above. As shown in FIG. 9, and aluminum anode 750 is formed over the aluminum cathode 702 and nanoporous aluminum oxide layer 710, to define a sensor 760.

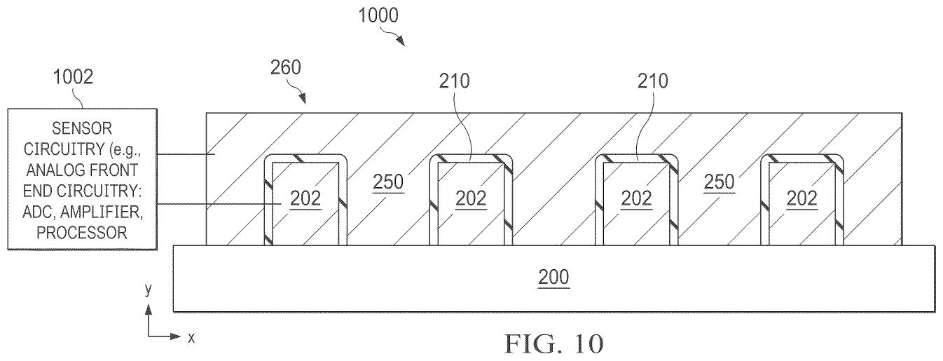

FIG. 10 shows an example sensor system 1000 including the example sensor 260 shown in FIGS. 5A-5B and sensor circuitry 1002 connected to the aluminum cathode 202 and aluminum anode 250. In some examples, sensor circuitry 1002 may include analog front end (AFE) circuitry, for example including an analog-to-digital converter (ADC), amplifier, and processor (e.g., embodied by a microcontroller, microprocessor, or other processor).

Although example embodiments have been described above, other variations and embodiments may be made from this disclosure without departing from the spirit and scope of these embodiments.

1. A method, comprising:
forming a metal cathode on a substrate;
anodizing an outer surface of the metal cathode to form an anodized porous layer;
forming a metal anode over the anodized porous layer;
wherein the anodized porous layer defines a dielectric layer between the metal anode and the metal cathode; and
wherein the metal anode, the anodized porous layer, and the metal cathode define a sensor.

2. The method of claim 1, comprising at least partially filling the anodized porous layer with at least one substance before forming the metal anode over the anodized porous layer.

3. The method of claim 1, comprising at least partially filling the anodized porous layer with at least one oxide before forming the metal anode over the anodized porous layer.

4. The method of claim 1, wherein:
the metal cathode comprises a three-dimensional metal cathode having a three-dimensional outer surface including different areas extending in different planes; and
the anodized porous layer comprises a three-dimensional porous layer.

5. The method of claim 4, wherein forming the three-dimensional metal cathode comprises forming a metal and selectively etching portions of the metal layer within an outer lateral perimeter of the metal layer to define a three-dimensional structure.

6. The method of claim 4, wherein forming the three-dimensional metal cathode comprises forming a metal and selectively etching portions of the metal layer to define an array of spaced apart cathode elements.

7. The method of claim 4, wherein a thickness of the three-dimensional metal cathode varies by at least 25% or at least 0.5 μm at different locations across a lateral footprint of the cathode.

8. The method of claim 1, wherein:
forming the metal cathode comprises forming an aluminum cathode; and
anodizing the outer surface of the metal cathode to form the anodized porous layer comprise anodizing an outer surface of the aluminum cathode to form a nanoporous aluminum oxide layer.

9. The method of claim 8, comprising at least partially filling the nanoporous aluminum oxide layer with at least one substance before forming the metal anode.

10. The method of claim 8, comprising at least partially filling the nanoporous aluminum oxide layer with at least one oxide before forming the metal anode.

11. The method of claim 8, comprising at least partially filling the nanoporous aluminum oxide layer with at least one of tin oxide, nickel oxide, or titanium oxide before forming the metal anode.

12. The method of claim 1, wherein:
forming the metal cathode comprises forming a three-dimensional aluminum cathode having a three-dimensional outer surface including different areas extending in different planes; and
anodizing the outer surface of the metal cathode to form the anodized porous layer comprise anodizing the three-dimensional outer surface of the three-dimensional aluminum cathode to form a three-dimensional nanoporous aluminum oxide layer.

13. The method of claim 12, wherein forming the three-dimensional aluminum cathode comprises:
depositing an aluminum layer; and
selectively etching portions of the aluminum layer within an outer lateral perimeter of the aluminum layer to define a three-dimensional structure.

14. A sensor, comprising:
a metal cathode formed on a substrate;
an anodized porous layer formed on an outer surface of the metal cathode; and
a metal anode formed over the anodized porous layer.

15. The sensor of claim 14, comprising at least one substance at least partially filling the anodized porous layer.

16. The sensor of claim 14, comprising at least one oxide at least partially filling the anodized porous layer.

17. The sensor of claim 14, wherein:
the metal cathode comprises a three-dimensional metal cathode having a three-dimensional outer surface including different areas extending in different planes; and
the anodized porous layer comprises a three-dimensional porous layer.

18. The sensor of claim 14, wherein:
the metal cathode comprises an aluminum cathode; and
the anodized porous layer comprises a nanoporous aluminum oxide layer.

19. The sensor of claim 14, wherein:
the metal cathode comprises a three-dimensional aluminum cathode having a three-dimensional outer surface including different areas extending in different planes; and
the anodized porous layer comprises a three-dimensional nanoporous aluminum oxide layer.

20. The sensor of claim 19, comprising at least one oxide at least partially filling the three-dimensional nanoporous aluminum oxide layer.

21. A sensor system, comprising:
a sensor, comprising:
an aluminum cathode formed on a substrate;
a nanoporous aluminum oxide layer formed on an outer surface of the aluminum cathode; and
an aluminum anode formed over the nanoporous aluminum oxide layer; and
sensor circuitry connected to the aluminum cathode and the aluminum anode to measure at least one of a resistance or a capacitance of the sensor.

21. A sensor system of claim 21, wherein:
the aluminum cathode comprises a three-dimensional structure having a three-dimensional outer surface including different areas extending in different planes; and
the nanoporous aluminum oxide layer has a three-dimensional structure.

23. The sensor system of claim 21, comprising at least one oxide at least partially filling the nanoporous aluminum oxide layer.
